(19) United States
(12) Patent Application Publication
(10) Pub. No.: US 2010/0286156 A1
(43) Pub. Date: Nov. 11, 2010

(54) Collyrium For The Treatment Of Conical Cornea With Cross-Linking Trans-Epithelial Technique

(76) Inventor: Roberto PINELLI, Brescia (IT)

Correspondence Address: Kirschstein, Israel, Schiffmiller & Pieroni, P.C., 425 FIFTH AVENUE, 5TH FLOOR, NEW YORK, NY, 10016-2223, US

(21) Appl. No.: 12/436,355

(22) Filed: May 6, 2009

(51) Int. Cl. A61K 31/525

(52) U.S. Cl. 514/251

(57) ABSTRACT

A collyrium for the treatment of patients suffering from conical cornea contains a riboflavin photosensitizing substance and a surface-active agent to assist penetration of the collyrium into the corneal epithelium. The collyrium does not require removal of the corneal epithelium, with the absence of pain for the patient, who does not need post treatment therapy, and who no longer complains of edema due to the prior art removal of the epithelium, thereby preserving the original corneal transparency.

### BACKGROUND OF THE INVENTION

The present invention relates to an improved collyrium for the treatment with cross-linking trans-epithelial technique of patients suffering from conical cornea.

In people suffering from conical cornea, the cornea is weaker and the normal links between the collagen fibers are much less numerous.

Conical cornea is a progressive pathology and bilateral in almost all cases, i.e. it affects both the eyes of the patient.

Within the aim of stiffening the corneal structure and assisting an increase of the links between the corneal collagen fibers (cross-linking) it is the practice to instill riboflavin in collyrium form onto the cornea and photosensitive it with a source of UVA rays.

Collyria presently known for this purpose require, for the treatment to be effective, that the corneal epithelium is removed, with consequent periods of corneal edema (opacity of the cornea) post-treatment and lengthy re-epithelization of the part treated.

### SUMMARY OF THE INVENTION

The aim of the present invention is to provide a new collyrium for the treatment of these patients that, unlike those existing, does not require removal of the corneal epithelium.

This aim is achieved with a collyrium containing the riboflavin photosensitizing substance and a surface-acting agent that assists penetration of the collyrium into the corneal epithelium.

Preferred embodiments result from the sub claims.

Other aims, advantages and characteristics result from the collyrium realized according to the invention, here described with reference to several examples of preparation of the same.

### BRIEF DESCRIPTION OF THE DRAWING

FIG. 1 depicts the administering procedure of the collyrium, devised according to the present invention.

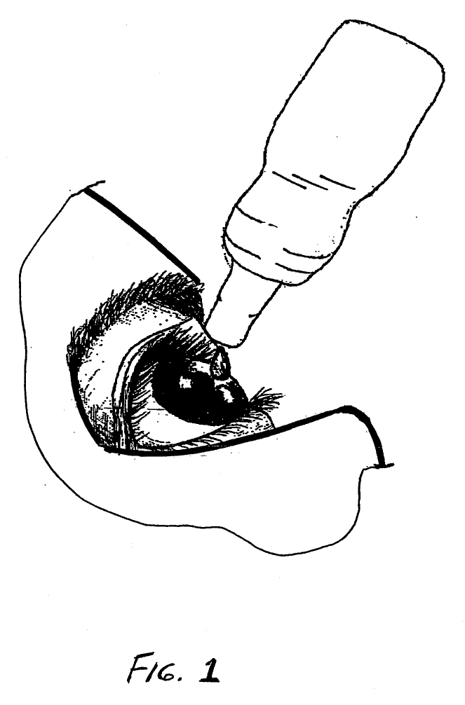

### DETAILED DESCRIPTION OF THE PREFERRED EMBODIMENTS

The collyrium of the present invention contains riboflavin, i.e. vitamin B₂ present in nature in food such as fruit and vegetables.

Riboflavin has photosensitizing capacities whereby, in the presence of ultraviolet rays, it accumulates energy and transfers it to surrounding means.

Therefore, when it is instilled onto the cornea (corneal stroma), which is composed essentially of water and collagen fibers, and then affected by a source of UVA rays (3 mW/cm² source and rays of a wavelength equal to 370 nm), the riboflavin first accumulates energy and then transfers it to the water molecules causing the hydrolysis thereof; the ions originating from this process (free radicals) link to the collagen fibers and reinforce the structure (cross-linking).

The traditional need to remove the corneal epithelium is overcome by the presence in the collyrium subject matter of the invention of a surface-active compound that has the task of making the riboflavin penetrate through the epithelium.

The preferred surface-active agent for the invention is in particular Benzal Chloride.

### Example

Hereinafter a formulation example of the collyrium is indicated prepared according to the present invention; the quantities are specified for 100 ml of solution:

150 mg riboflavin-5-sodium phosphate (powder)
440 mg Sodium Chloride (powder)
900 mg Sterile water (liquid)
20 mg Benzal Chloride

1-6. (canceled)

7. A collyrium for the treatment of conical cornea by a cross-linking trans-epithelial technique, comprising: . a riboflavin provided with photosensitizing properties; and a surface-active compound that assists penetration of the riboflavin into a corneal epithelium.

8. The collyrium of claim 7, wherein the surface-active agent is benzal chloride.

9. The collyrium of claim 1, comprising, for 100 ml of solution:
150 mg riboflavin-5-sodium phosphate in powdered form,
440 mg sodium chloride in powdered form,
900 mg sterile liquid water, and
20 mg benzal chloride.

10. A method of treatment of a conical cornea by a cross-linking trans-epithelial technique, comprising the steps of: providing a riboflavin with photosensitizing properties; adding to the riboflavin a surface-active compound to form a collyrium; and applying the collyrium to a patient's conical cornea, the surface-active compound assisting penetration of the riboflavin into a corneal epithelium.

11. The method of claim 10, wherein the photo-sensitization of the riboflavin occurs with the assistance of ultraviolet rays.

12. The method of claim 11, wherein the photo-sensitization is performed with a source of ultraviolet rays of 3 mW/cm² source and rays of a wavelength equal to 370 nm.
